(19) United States
(12) Patent Application Publication
(10) Pub. No.: US 2021/0147434 A1
(43) Pub. Date: May 20, 2021

(54) PROCESS FOR THE PREPARATION OF MORPHINANE COMPOUNDS

(71) Applicant: Saneca Pharmaceutical a.s., Hlohovec (SK)

(72) Inventors: Ján GASPAR, Bratislava-Dúbravka (SK); Richard HERCEK, Hlohovec (SK); Miroslav KAVALA, Leopoldov (SK)

(73) Assignee: Saneca Pharmaceutical a.s., Hlohovec (SK)

(21) Appl. No.: 16/625,436

(22) PCT Filed: Jun. 29, 2018
(86) PCT No.: PCT/SK2018/000003

§ 371 Date: Dec. 20, 2019

(30) Foreign Application Priority Data
Jul. 4, 2017 (SK) PP 00059-2017

(51) Int. Cl. C07D 489/08; B01J 27/08; B01J 31/02

(52) U.S. Cl. CPC C07D 489/08; B01J 27/08; B01J 2231/641; B01J 31/0268; B01J 2531/002; B01J 31/0239

(57) ABSTRACT

The invention describes the process of catalytic O-demethylation of 3-methoxymorphinane compounds using boron tribromide. Addition of catalysts reduces the reaction time, improves reacting the substrate to give the product in very good purity and yield. The process can be used, for example, for the preparation of oxycodone, oxymorphone, naltrexone, naloxone and nalbuphine from their respective O-methyl derivatives.

### FIELD OF THE INVENTION

The invention is from the field of pharmaceutical manufacturing. It relates to the preparation of intermediates or active pharmaceutical ingredients (API's) based on morphinane compounds. The invention is directed to the synthesis of 3-hydroxymorphinane compounds by O-demethylation of 3-methoxymorphinane compounds according to the following scheme:

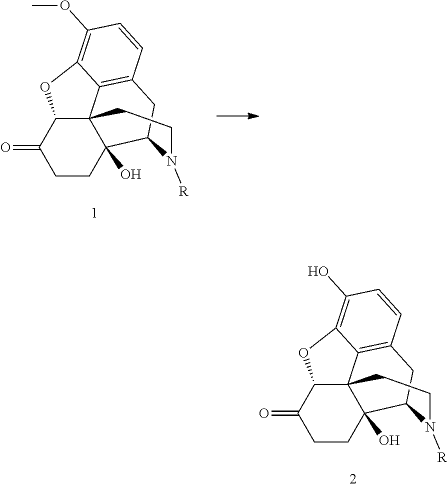

These derivatives affect the receptors of the central nervous system, and as such can be used as medicines for pain and for reducing psychological dependence in patients addicted to drugs. The most commonly used morphinane derivatives in this area include, for example, oxycodone, oxymorphone, naloxone, naltrexone, and nalbuphine.

### BACKGROUND OF THE INVENTION

The most commonly used O-demethylating agents used for the preparation of 3-hydroxymorphinane derivatives include hydrobromic acid, boron tribromide, and the methanesulphonic acid/methionine system, as described in the literature. The yields of these demethylations range from 30 to 80%, depending on the morphinane compound itself. The dealkylating agents used in literature are summarized in papers Tetrahedron, <57, 2005, 7833-7863 and Synthesis 1983, 249-283.

International application WO 2013/050748 A2 describes the preparation of buprenophine. The authors use a mixture of mercaptan and a strong organic base at a higher temperature for O-demethylation. In this way, the authors obtained the demethylated product in yields from 70 to 85%.

U.S. Pat. No. 4,667,037 describes O-dealkylation using HBr, HCl, and HI with addition of boric acid or various inorganic salts. In this way, the authors obtained the respective hydroxy derivatives in yields from 65 to 85%.

Patent CN 103113378 describes the preparation of oxymorphone hydrochloride by O-demethylation of oxycodone. The authors describe O-demethylation using amino acids in an acidic environment, thereby obtaining high purity product in yields of 70-80%.

U.S. Pat. No. 5,071,985 describes the preparation of morphinane derivatives by O-demethylation of 3-methoxy derivatives with methanesulphonic acid or trifluoromethanesulphonic acid in the presence of a sulphide (methionine). Depending on the starting morphinane, the authors obtained product in yields of 60-90%.

A common feature of the above methods is a varying extent of the starting substrate degradation during the ongoing O-demethylation, which results in a decrease in product yield and quality.

In this regard, the use of boron tribromide (BBr₃) seems to be more advantageous because O-demethylation takes place more selectively, at higher purity, and with better yields. Its advantage consists in the high reactivity of the agent under mild conditions, which also excludes O-demethylation in strongly acidic or basic environment and at higher temperatures. BBr₃ selectively demethylates methyl ethers, while not affecting double bonds or ester groups present in the molecule. Demethylation typically takes place in aprotic solvents (e.g., dichloromethane, chloroform, chlorobenzene, toluene, pentane, etc.) at room temperature. The processing of the reaction mixture includes hydrolysis with water and precipitation of the product in the form of base after pH adjustment or extracting the product into a suitable solvent after pH adjustment. Depending on the conditions, a high quality product is obtained using this method in yields of 70-98%.

International application WO 1999/02529A1 describes preparation of naltrexone by O-demethylation of 3-methoxy derivative using BBr₃ in dichloromethane. The authors obtained product in a yield of 98%.

International application WO 2007/137785A2 describes preparation of morphinane derivatives by O-demethylation using BBr₃ in aprotic solvents. The authors obtained product in yields of 60-90%.

International application WO 2009/111162 describes preparation of oxymorphone by O-demethylation of oxycodone with BBr₃ in dichloromethane. The authors focus on studying the effect of pH on the hydrolysis of intermediates. The authors isolated the product in the form of a crude base in a yield of 78%.

At present, the approach starts to prevail seeking to reduce or eliminate the use of chlorinated solvents, and it is therefore necessary to test the above-mentioned O-demethylation procedure in non-halogenated solvents. Because BBr₃ is highly reactive, only an inert solvent such as toluene can be used as the reaction medium. If oxycodone base is reacted in toluene with 3.3 eq. of BBr₃ according to the standard reaction protocol, then 23 hours later about 10% of unreacted oxycodone still remains in the reaction mixture at room temperature.

Catalytic O-demethylation using BBr₃ is not yet known in literature. O-demethylations with HBr with addition of phase catalysts are well-known. (Landini, D.; Montanari, F.; Rolla, F. Synthesis 1978, 771-773, Brindaban C. R.; Sanjay B. Org. Prep. Proced. Int. 28, (4), 1996, 371-409).

### SUMMARY OF THE INVENTION

Boron tribromide (BBr₃) is one of the most advantageous agents for O-demethylation of methyl ethers. In addition to toxicity and poor handling of the agent, another disadvantage of the prior art processes is the work in halogenated solvents. If a non-halogenated solvent compatible with BBr₃ is used, e.g., toluene, a long reaction time (sometimes up to 68 hours) is observed to obtain conversion of the starting material of less than 5%, especially if the substrate is not well soluble in the reaction medium. Due to the low solubility of the oxycodone base in toluene and the fact that the reaction mixture remains heterogeneous throughout the reaction, the effect of catalysts on the reaction was examined. Surprisingly, it was found that by adding the catalyst, the reaction is accelerated without negatively affecting the yield and product quality. On the contrary, the isolated product contained less unreacted substrate.

The invention addresses O-demethylation of morphinane compounds using BBr₃ with addition of catalysts and their effect on the reaction rate.

### DETAILED DESCRIPTION OF THE INVENTION

This invention is defined in the appended claims.

Process for the preparation of a morphinane compound (2) from a morphinane compound (1) or salts thereof is carried out according to the following scheme,

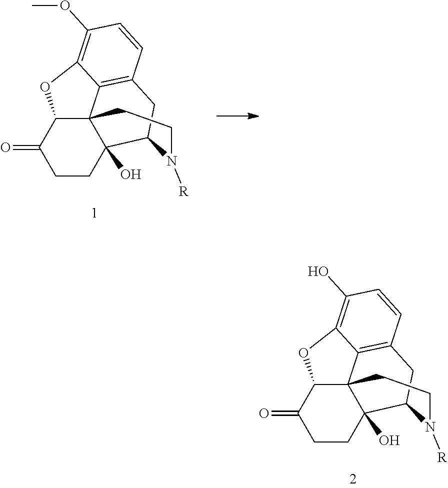

where R in the morphinane compounds (1) and (2) is hydrogen, hydrocarbyl or substituted hydrocarbyl, e.g., methyl, ethyl, propyl, allyl, cyclopropylmethyl, or cyclobutylmethyl, wherein the morphinane compound (1) may be the following compounds: 4,5α-epoxy-14-hydroxy-3-methoxymorphinan-6-one (noroxycodone) or 4,5α-epoxy-14-hydroxy-3-methoxy-17-methyl-morphinan-6-one (oxycodone) or 4,5α-epoxy-14-hydroxy-3-methoxy-17-ethylmorphinan-6-one or 4,5α-epoxy-14-hydroxy-3-methoxy-17-propylmorphinan-6-one or 4,5α-epoxy-14-hydroxy-3-methoxy-17-allylmorphinan-6-one (3-methoxynaloxone) or 4,5α-epoxy-14-hydroxy-3-methoxy-17-cyclopropylmethylmorphinan-6-one (3-methoxynaltrexone) or 4,5α-epoxy-14-hydroxy-3-methoxy-17-cyclobutylmethylmorphinan-6-one (3-methoxynalbuphone), and the morphinane compound (2) is 4,5α-epoxy-3,14-dihydroxymorphinan-6-one (noroxymorphone) or 4,5α-epoxy-3,14-dihydroxy-17-methylmorphinan-6-one (oxymorphone) or 4,5α-epoxy-3,14-dihydroxy-17-ethylmorphinan-6-one or 4,5α-epoxy-3,14-dihydroxy-17-propylmorphinan-6-one or 4,5α-epoxy-3,14-dihydroxy-17-allylmorphinan-6-one (naloxone) or 4,5α-epoxy-3,14-dihydroxy-17-cyclopropylmethylmorphinan-6-one (naltrexone) or 4,5α-epoxy-3,14-dihydroxy-17-cyclobutylmethylmorphinan-6-one (nalbuphone). A reaction of a morphinane compound of Formula (1) or salts thereof with boron tribromide (BBr₃) takes place in the presence of catalysts in an aprotic solvent selected from a group consisting of benzene, toluene, o-xylene, m-xylene, p-xylene, chlorobenzene, dichloromethane, chloroform, or mixtures thereof. BBr₃ is typically added to the mixture in excess in relation to the morphinane compound (1) at a reduced temperature of 5 to 15° C., then the reaction mixture is allowed to warm to room temperature. In general, BBr₃ is added slowly to the reaction mixture consisting of a morphinane compound (1) and a catalyst. In certain cases, it is possible to add a solution of morphinane compound (1) and a catalyst in an aprotic solvent to a solution of BBr₃ in this solvent at reduced temperature. Typically, an excess of the agent in relation to the morphinane compound (1) is used, of more than 1 molar equivalent, preferably 3 to 4 molar equivalents.

The catalyst used is an inorganic iodide or a quaternary iminium or phosphonium compound of the following formula:

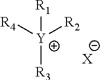

The catalyst used may be, e.g., lithium iodide, sodium iodide, potassium iodide, or an iminium or phosphonium compound, where Y is N or P, and R₁ is hydrogen, hydrocarbyl or substituted hydrocarbyl, aryl, or cycloalkyl, R₂ is hydrogen, hydrocarbyl or substituted hydrocarbyl, aryl, or cycloalkyl, R₃ is hydrogen, hydrocarbyl or substituted hydrocarbyl, aryl, or cycloalkyl, R₄ is hydrogen, hydrocarbyl or substituted hydrocarbyl, aryl, or cycloalkyl, and X is F, Cl, Br, I, sulphate, sulphite, hydrogensulphate, hydrogensulphite, nitrate, nitrite, phosphate, hydrogenphosphate, or dihydrogenphosphate.

The catalyst is used in an amount of 0.1 to 1 molar equivalent in relation to the morphinane compound 1, preferably 0.3 to 0.5 molar equivalent, wherein it is preferable to use tetrabutylammonium chloride, tetrabutylammonium bromide (TBAB), tetrabutylammonium iodide (TBAI), benzyltriethylammonium chloride, benzyltriethylammonium bromide (TEBA), benzyltriethylammonium iodide, benzyltrimethylammonium chloride, benzyltrimethylammonium bromide, benzyltrimethylammonium iodide, cetyltriethylammonium chloride, cetyltriethylammonium bromide (CTAB), or cetyltriethylammonium iodide.

The reaction takes place at a temperature of 15 to 25° C. for 4 to 15 hours, then it is processed by hydrolysis in an aqueous environment. Subsequently, morphinane compound (2) is isolated from the aqueous phase after pH adjustment by extraction to a suitable solvent or by precipitating the crude base from the mixture using an inorganic base (NH₄OH, NaOH, or KOH).

TABLE 1
Preparation of oxymorphone (OM), comparison of the effect of adding
catalyst on the rate of O-demethylation of oxycodone (OC)

| | | | HPLC area % | | HPLC area % | | HPLC area % | | HPLC area % | |
|---|---|---|---|---|---|---|---|---|---|---|
| | Agent | | After 1 h | | After 3 h | | After 6 h | | After 23 h | |
| Solvent | (excess) | Catalyst | OM | OC | OM | OC | OM | OC | OM | OC |
| Toluene | BBr₃ | — | — | — | 67.8 | 31.8 | — | — | 86.5 | 12.7 |
| | (3.33 eq.) | | | | | | | | | |
| Toluene | BBr₃ | NaI | 21.8 | 77.6 | 88.9 | 10.7 | 90.2 | 5.9 | 97.1 | 2.8 |
| | (3.33 eq.) | (0.5 eq.) | | | | | | | | |
| Toluene | BBr₃ | KI | 73.2 | 25.9 | 78.6 | 20.8 | 83.1 | 15.8 | 94.1 | 4.7 |
| | (3.33 eq.) | (0.5 eq.) | | | | | | | | |
| Toluene | BBr₃ | TBAI | 86.6 | 12.5 | 90.9 | 8.2 | 93.2 | 5.8 | 96.9 | 1.3 |
| | (3.33 eq.) | (0.5 eq.) | | | | | | | | |
| Toluene*^{a} | BBr₃ | TBAB | 92.6 | 6.8 | 97.5 | 2.3 | 99.8 | 0.1 | — | — |
| | (3.33 eq.) | (0.5 eq.) | | | | | | | | |
| Toluene | BBr₃ | CTAB | 77.9 | 21.5 | 82.3 | 17.1 | 85.6 | 13.8 | 92.1 | 7.1 |
| | (3.33 eq.) | (0.1 eq.) | | | | | | | | |
| Toluene | BBr₃ | THPB | 97.9 | 1.9 | 98.43 | 1.2 | 98.8 | 0.9 | — | — |
| | (3.33 eq.) | (0.1 eq.) | | | | | | | | |

*^{a}conversion <1 area % after 5 h

The crude base of morphinane compound (2) obtained can then be converted to the corresponding salt by adding the respective acid to the crude base. For this purpose, hydrochloric, sulfuric, phosphoric, or tartaric etc. acid can be used.

### BRIEF DESCRIPTION OF THE DRAWINGS

FIG. 1 shows the effect of the catalyst on the O-demethylation rate of oxycodone on oxymorphone through the action of BBr₃.

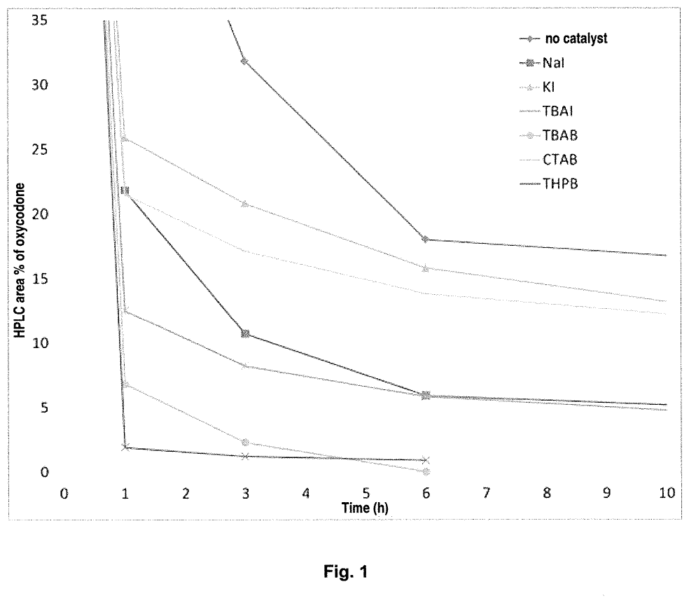

### EXAMPLES

The examples provided are intended to illustrate the invention.

### Example 1: Preparation of Oxymorphone by O-Demethylation of Oxycodone in the Presence of NaI

The oxycodone base (5.0 g) is weighed together with sodium iodide (0.5 eq.) and toluene (75 mL) is added. The suspended mixture is cooled to 0° C. BBr₃ (3.3 eq.) is added dropwise over 15 minutes by means of a dropping funnel with continuous stirring. During the addition, the temperature is maintained between 5 and 15° C. The reaction mixture is allowed to warm to room temperature after the addition, and the stirring continues. After 23 hours, 2.0 area % of the starting material remains in the reaction mixture. The reaction mixture is hydrolyzed with water and the oxymorphone is isolated by precipitation or extraction into an organic solvent after pH adjustment to >7.

### Example 2: Preparation of Oxymorphone by O-Demethylation of Oxycodone in the Presence of KI

The oxycodone base (5.0 g) is weighed together with potassium iodide (0.5 eq.) and toluene (75 mL) is added. The suspended mixture is cooled to 0° C. BBr₃ (3.3 eq.) is added dropwise over 15 minutes by means of a dropping funnel with continuous stirring. During the addition, the temperature is maintained between 5 and 15° C. The reaction mixture is allowed to warm to room temperature after the addition, and the stirring continues. After 23 hours, 4.1 area % of the starting material remains in the reaction mixture. The reaction mixture is hydrolyzed with water and the oxymorphone is isolated by precipitation or extraction into an organic solvent after pH adjustment to >7.

### Example 3: Preparation of Oxymorphone by O-Demethylation of Oxycodone in the Presence of TBAI

The oxycodone base (5.0 g) is weighed together with TBAI (0.5 eq.) and toluene (75 mL) is added. The suspended mixture is cooled to 0° C. BBr₃ (3.3 eq.) is added dropwise over 15 minutes by means of a dropping funnel with continuous stirring. During the addition, the temperature is maintained between 5 and 15° C. The reaction mixture is allowed to warm to room temperature after the addition, and the stirring continues. After 8 hours, 3.8 area % of the starting material remains in the reaction mixture. The reaction mixture is hydrolyzed with water and the oxymorphone is isolated by precipitation or extraction into an organic solvent after pH adjustment to >7.

### Example 4: Preparation of Oxymorphone by O-Demethylation of Oxycodone in the Presence of TBAB

The oxycodone base (5.0 g) is weighed together with TBAB (0.5 eq.) and toluene (75 mL) is added. The suspended mixture is cooled to 0° C. BBr₃ (3.6 eq.) is added dropwise over 15 minutes by means of a dropping funnel with continuous stirring. During the addition, the temperature is maintained between 5 and 15° C. The reaction mixture is allowed to warm to room temperature after the addition, and the stirring continues. After 5 hours, <1 area % of the starting material remains in the reaction mixture. The reaction mixture is hydrolyzed with water and the oxymorphone is isolated by precipitation or extraction into an organic solvent after pH adjustment to >7.

### Example 5: Preparation of Oxymorphone by O-Demethylation in the Presence of CTAB

The oxycodone base (5.0 g) is weighed together with CTAB (0.1 eq.) and toluene (75 mL) is added. The suspended mixture is cooled to 0° C. BBr₃ (3.3 eq.) is added dropwise over 15 minutes by means of a dropping funnel with continuous stirring. During the addition, the temperature is maintained between 5 and 15° C. The reaction mixture is allowed to warm to room temperature after the addition, and the stirring continues. After 23 hours, 5.9 area % of the starting material remains in the reaction mixture. The reaction mixture is hydrolyzed with water and the oxymorphone is isolated by precipitation or extraction into an organic solvent after pH adjustment to >7.

### Example 6: Preparation of Oxymorphone by O-Demethylation in the Presence of TEBA

The oxycodone base (5.0 g) is weighed together with TEBA (0.3 eq.) and toluene (75 mL) is added. The suspended mixture is cooled to 0° C. BBr₃ (3.6 eq.) is added dropwise over 15 minutes by means of a dropping funnel with continuous stirring. During the addition, the temperature is maintained between 5 and 15° C. The reaction mixture is allowed to warm to room temperature after the addition, and the stirring continues. After 8 hours, less than 5 area % of the starting material remains in the reaction mixture. The reaction mixture is hydrolyzed with water and the oxymorphone is isolated by precipitation or extraction into an organic solvent after pH adjustment to >7.

### Example 7: Preparation of Oxymorphone by O-Demethylation of Oxycodone in the Presence of Tributylhexadecylphosphonium Bromide (THPB)

The oxycodone base (5.0 g) is weighed together with THPB (0.1 eq.) and toluene (75 mL) is added. The suspended mixture is cooled to 0° C. BBr₃ (4.0 eq.) is added dropwise over 15 minutes by means of a dropping funnel with continuous stirring. During the addition, the temperature is maintained between 5 and 15° C. The reaction mixture is allowed to warm to room temperature after the addition, and the stirring continues. After 5 hours, <1 area % of the starting material remains in the reaction mixture. The reaction mixture is hydrolyzed with water and the oxymorphone is isolated by precipitation or extraction into an organic solvent after pH adjustment to >7.

### Example 8: Preparation of Noroxymorphone by O-Demethylation of Noroxycodone in the Presence of Tetrabutylphosphonium Bromide (TPB-Br)

The noroxycodone (5.0 g) is weighed together with TPB-Br (0.8 eq.) and chlorobenzene (75 mL) is added. The suspended mixture is cooled to 0° C. BBr₃ (3.8 eq.) is added dropwise over 15 minutes by means of a dropping funnel with continuous stirring. During the addition, the temperature is maintained between 5 and 15° C. The reaction mixture is allowed to warm to room temperature after the addition, and the stirring continues. After 6 hours, less than 2 area % of the starting material remains in the reaction mixture. The reaction mixture is hydrolyzed with water and the noroxymorphone is isolated by precipitation or extraction into an organic solvent after pH adjustment to >7.

### Example 9: Preparation of Naltrexone by O-Demethylation of 3-Methoxynaltrexone in the Presence of TBAB

3-methoxznaltrexone (5.0 g) is weighed together with TBAB (0.1 eq.) and toluene (75 mL) is added. The suspended mixture is cooled to 0° C. BBr₃ (3.1 eq.) is added dropwise over 15 minutes by means of a dropping funnel with continuous stirring. During the addition, the temperature is maintained between 5 and 15° C. The reaction mixture is allowed to warm to room temperature after the addition, and the stirring continues. After 3 hours, less than 1 area % of the starting material remains in the reaction mixture. The reaction mixture is hydrolyzed with water and the naltrexone is isolated by precipitation or extraction into an organic solvent after pH adjustment to >7.

### Example 10: Preparation of Naloxone by O-Demethylation of 3-Methoxynaloxone in the Presence of TBAB

3-methoxynaloxone (5.0 g) is weighed together with TBAB (0.2 eq.) and toluene (75 mL) is added. The suspended mixture is cooled to 0° C. BBr₃ (3.2 eq.) is added dropwise over 15 minutes by means of a dropping funnel with continuous stirring. During the addition, the temperature is maintained between 5 and 15° C. The reaction mixture is allowed to warm to room temperature after the addition, and the stirring continues. After 3 hours, less than 1 area % of the starting material remains in the reaction mixture. The reaction mixture is hydrolyzed with water and the naloxone is isolated by precipitation or extraction into an organic solvent after pH adjustment to >7.

1. A process for the preparation of a morphinane compound (2) from a morphinane compound (1) or salts thereof, the process comprising:
(i) mixing the morphinane compound (1) with a catalyst in an aprotic organic solvent; and
(ii) adding boron tribromide to the mixture of (i), wherein substituent R in the morphinane compound (1) and the morphinane compound (2) is selected from hydrogen, hydrocarbyl or substituted hydrocarbyl, according to a scheme:

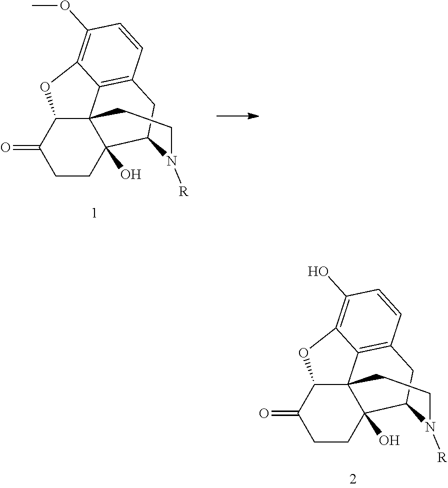

2. The process according to claim 1, wherein substituent R in the morphinane compound (1) and the morphinane compound (2) is hydrogen, methyl, ethyl, propyl, allyl, cyclopropylmethyl or cyclobutylmethyl, and the aprotic organic solvent is selected from the group consisting of benzene, toluene, o-xylene, m-xylene, p-xylene, chlorobenzene, dichloromethane, chloroform, and mixtures thereof.

3. The process according to claim 1, wherein the morphinane compound (1) is 4,5α-epoxy-14-hydroxy-3-methoxymorphinan-6-one (noroxycodone), 4,5α-epoxy-14-hydroxy-3-methoxy-17-methylmorphinan-6-one (oxycodone), 4,5α-epoxy-14-hydroxy-3-methoxy-17-ethylmorphinan-6-one, or 4,5α-epoxy-14-hydroxy-3-methoxy-17-propyl-morphinan-6-one, 4,5α-epoxy-14-hydroxy-3-methoxy-17-allylmorphinan-6-one (3-methoxynaloxone), 4,5α-epoxy-14-hydroxy-3-methoxy-17-cyclopropylmethyl-morphinan-6-one (3-methoxynaltrexone) or 4,5α-epoxy-14-hydroxy-3-methoxy-17-cyclobutylmethyl-morphinan-6-one (3-methoxynalbuphone); and the morphinane compound (2) is 4,5α-epoxy-3,14-dihydroxymorphinan-6-one (noroxymorphone), 4,5α-epoxy-3,14-dihydroxy-17-methyl-morphinan-6-one (oxymorphone), 4,5α-epoxy-3,14-dihydroxy-17-ethylmorphinan-6-one, 4,5α-epoxy-3,14-dihydroxy-n-propyl-morphinan-O-one, 4,5α-epoxy-3,14-dihydroxy-17-allylmorphinan-6-one (naloxone), 4,5α-epoxy-3,14-dihydroxy-17-cyclopropylmethyl-morphinan-6-one (naltrexone) or 4,5α-epoxy-3,14-dihydroxy-17-cyclobutylmethyl-morphinan-6-one (nalbuphone).

4. The process according to claim 1, wherein the boron tribromide is added in excess in relation to the morphinane compound (1).

5. The process according to claim 4, wherein the excess of the boron tribromide is more than 1 molar equivalent.

6. The process according to claim 1, wherein the catalyst is (i) an inorganic iodide; or
(ii) a quaternary iminium or phosphonium compound of formula: 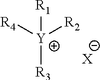
wherein Y is N or P;
R₁ is hydrogen, hydrocarbyl or substituted hydrocarbyl, aryl or cycloalkyl;
R₂ is hydrogen, hydrocarbyl or substituted hydrocarbyl, aryl or cycloalkyl;
R₃ is hydrogen, hydrocarbyl or substituted hydrocarbyl, aryl or cycloalkyl;
R₄ is hydrogen, hydrocarbyl or substituted hydrocarbyl, aryl or cycloalkyl; and
X is F, Cl, Br, I, sulphate, sulphite, hydrogensulphate, hydrogensulphite, nitrate, nitrite, phosphate, hydrogenphosphate or dihydrogenphosphate.

7. The process according to claim 6, wherein the inorganic iodide catalyst is lithium iodide, sodium iodide, or potassium iodide.

8. The process according to claim 6, wherein the catalyst is in an amount of 0.1 to 1 molar equivalent in relation to the morphinane compound (1).

9. The process according to claim 6, wherein the catalyst is tetrabutylammonium chloride, tetrabutylammonium bromide, tetrabutylammonium iodide, benzyltriethylammonium chloride, benzyltriethylammonium bromide, benzyltriethylammonium iodide, benzyltrimethylammonium chloride, benzyltrimethylammonium bromide, benzyltrimethylammonium iodide, cetyltriethylammonium chloride, cetyltriethylammonium bromide or cetyltriethylammonium iodide.

10. A process for catalytic O-demethylation of a morphinane compound having a structure of formula 1 (morphinane compound (1)), wherein substituent R is selected from hydrogen, hydrocarbyl or substituted hydrocarbyl 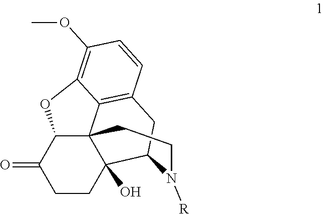
the process comprising:
(i) mixing the morphinane compound (1) with a catalyst in an aprotic organic solvent; and
(ii) adding boron tribromide to the mixture of (i).

11. The process according to claim 10, wherein R in the morphinane compound (1) is hydrogen, methyl, ethyl, propyl, allyl, cyclopropylmethyl, or cyclobutylmethyl, and the aprotic organic solvent is selected from the group consisting of benzene, toluene, o-xylene, m-xylene, p-xylene, chlorobenzene, dichloromethane, chloroform, and mixtures thereof.

12. The process according to claim 10, wherein the morphinane compound (1) is 4,5α-epoxy-14-hydroxy-3-methoxymorphinan-6-one (noroxycodone), 4,5α-epoxy-14-hydroxy-3-methoxy-17-methylmorphinan-6-one (oxycodone), or 4,5α-epoxy-14-hydroxy-3-methoxy-17-ethylmorphinan-6-one, 4,5α-epoxy-14-hydroxy-3-methoxy-17-propyl-morphinan-6-one, 4,5α-epoxy-14-hydroxy-3-methoxy-17-allylmorphinan-6-one (3-methoxynaloxone), 4,5α-epoxy-14-hydroxy-3-methoxy-17-cyclopropylmethylmorphinan-6-one (3-methoxynaltrexone) or 4,5α-epoxy-14-hydroxy-3-methoxy-17-cyclobutylmethyl-morphinan-6-one (3-methoxynalbuphone).

13. The process according to claim 10, wherein the boron tribromide is added in excess in relation to the morphinane compound (1).

14. The process according to claim 13, wherein the excess of the boron tribromide is more than 1 molar equivalent.

15. The process according to claim 10, wherein the catalyst is (i) an inorganic iodide or (ii) a quaternary iminium or phosphonium compound of formula: 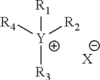
wherein Y is N or P;
R₁ is hydrogen, hydrocarbyl or substituted hydrocarbyl, aryl or cycloalkyl;
R₂ is hydrogen, hydrocarbyl or substituted hydrocarbyl, aryl or cycloalkyl;
R₃ is hydrogen, hydrocarbyl or substituted hydrocarbyl, aryl or cycloalkyl;
R₄ is hydrogen, hydrocarbyl or substituted hydrocarbyl, aryl or cycloalkyl; and
X is F, Cl, Br, I, sulphate, sulphite, hydrogensulphate, hydrogensulphite, nitrate, nitrite, phosphate, hydrogenphosphate or dihydrogenphosphate.

16. The process according to claim 15, wherein the inorganic iodide catalyst is lithium iodide, sodium iodide, or potassium iodide.

17. The process according to claim 15, wherein the catalyst is in an amount of 0.1 to 1 molar equivalent in relation to the morphinane compound (1).

18. The process according to claim 15, wherein the catalyst is tetrabutylammonium chloride, tetrabutylammonium bromide, tetrabutylammonium iodide, benzyltriethylammonium chloride, benzyltriethylammonium bromide, benzyltriethylammonium iodide, benzyltrimethylammonium chloride, benzyltrimethylammonium bromide, benzyltrimethylammonium iodide, cetyltriethylammonium chloride, cetyltriethylammonium bromide or cetyltriethylammonium iodide.

19. The process according to claim 10, wherein the catalytic O-demethylation of the morphinane compound (1) prepares a morphinane compound having a structure of formula 2 (morphinane compound (2)), wherein substituent R is selected from hydrogen, hydrocarbyl or substituted hydrocarbyl:

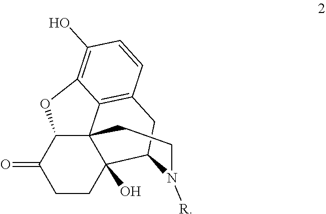

20. The process according to claim 19, wherein substituent R in the morphinane compound (2) is hydrogen, methyl, ethyl, propyl, allyl, cyclopropylmethyl or cyclobutylmethyl, and the aprotic organic solvent is selected from the group consisting of benzene, toluene, o-xylene, m-xylene, p-xylene, chlorobenzene, dichloromethane, chloroform, and mixtures thereof.

21. The process according to claim 19, wherein the morphinane compound (2) is 4,5α-epoxy-3,14-dihydroxymorphinan-6-one (noroxymorphone), 4,5α-epoxy-3,14-dihydroxy-17-methylmorphinan-6-one (oxy morph one), 4,5α-epoxy-3,14-dihydroxy-17-ethylmorphinan-6-one, or 4,5α-epoxy-3,14-dihydroxy-17-propyl morphinan-6-one, or 4,5α-epoxy-3,14-dihydroxy-17-allylmorphinan-6-one (naloxone), 4,5α-epoxy-3,14-dihydroxy-17-cyclopropylmethylmorphinan-6-one (naltrexone) or 4,5α-epoxy-3,14-dihydroxy-17-cyclobutylmethylmorphinan-6-one (nalbuphone).

22-27. (canceled)

28. The process according to claim 4, wherein the excess of the boron tribromide is 3 to 4 molar equivalents.

29. The process according to claim 6, wherein the catalyst is in an amount of 0.3 to 0.5 molar equivalent in relation to the morphinane compound (1).

30. The process according to claim 13, wherein the excess of the boron tribromide is 3 to 4 molar equivalents.

31. The process according to claim 15, wherein the catalyst is in an amount of 0.3 to 0.5 molar equivalent in relation to the morphinane compound (1).
